(19) United States
(12) Patent Application Publication
(10) Pub. No.: US 2003/0225387 A1
(43) Pub. Date: Dec. 4, 2003

(54) Absorbent ostomy pad system

(76) Inventor: Ricky L. Zedlitz, Whitehouse, TX (US)

Correspondence Address: Ronald B. Sefrna, Sefrna & Associates, P.O. Box 567, Tyler, TX, 75710-0567, US

(21) Appl. No.: 10/448,558

(22) Filed: May 30, 2003

Related U.S. Application Data
(60) Provisional application No. 60/385,029, filed on May 31, 2002.

(51) Int. Cl. A61F 13/15; A61F 13/20

(52) U.S. Cl. 604/385.03

(57) ABSTRACT

An absorbent ostomy pad system for absorbing and containing waste emitted from a stoma in the body of an ostomy patient includes a highly absorbent thin body, preferably formed of one or more layers of cellulose fluff with a liquid absorbent polymer dispersed through the fluff, a permeable sheet disposed on one face of the absorbent body, an impermeable sheet disposed on the opposite face of the absorbent body and connected to the first sheet, to enclose the absorbent body between the first and second sheets, and attachment means for removably holding the pad over the stoma. The attachment means may be an adhesive, or may be an elastic band to encircle the patient's body over the pad. Odor control agents such as baking soda may be included in the absorbent body of the pad.

### RELATED APPLICATION

The present application is related to and claims the benefit of U.S. Provisional Patent Application Serial No. 60/385,029, filed in the United States Patent and Trademark Office on May 31, 2002, and titled, “Absorbent Ostomy Pad System”.

### FIELD OF THE INVENTION

The present invention generally relates to devices for the collection of bodily wastes, and in its currently preferred embodiments more specifically relates to devices for the absorption and collection of body fluids and wastes emitted from a stoma associated with a colostomy, ileostomy, or urostomy.

### BACKGROUND OF THE INVENTION

In the medical procedure generally referred to as an ostomy, a portion of the patient's intestinal or urinary tract is removed and the bodily wastes conveyed through those organs are routed to the exterior of the patients body through a tube or stoma, typically located in the patient's abdominal region. Traditionally and conventionally, bodily wastes emitted from the body through the stoma have been collected in a receptacle or bag, often referred to as an ostomy appliance, which has an opening that is registered with the stoma to receive waste materials therefrom into the interior of the bag. Such ostomy appliances are connected to the body with an adhesive intended to seal a flange surrounding the opening of the bag to the skin around the stoma, or with a belt to hold the appliance in place, or both. When the bags are filled they may be flushed, for reuse, or disposed of.

When the ostomy procedure was first developed, the ostomy appliances, while generally effective, were relatively crude, difficult to use, and fraught with numerous disadvantages. Subsequently a number of improvements have been made, and developments in this field have focused on devising and providing improved ostomy appliances. Nevertheless, the same basic collection bag approach has been essentially universally employed for the collection and management of bodily wastes emitted from an ostomy stoma. Despite the numerous improvements that have been made, the use of ostomy bags presents problems and difficulties for many ostomy patients. Some patients develop sensitivity to the adhesives and/or to the plastic materials from which the bags are constructed and exhibit dermatological problems in the areas covered by the bags. Further, the ostomy bags remain somewhat bulky and cumbersome and limit physical activity, creating problems for users with active physical lifestyles. Ostomy appliances and the related supplies and materials are also costly, and the ongoing expense can be difficult for many ostomy patients to bear. Flushing and cleaning of ostomy bags can present problems for users, especially in public restrooms.

The use of absorbent pads for limited purposes is known in the prior art, but such uses have been predominantly directed toward providing a mildly absorbent pad around the stoma and between the ostomy bag and the skin or a patient, to absorb fluids that may leak through an incomplete seal between the ostomy bag and the skin. In general, the prior art approaches to collection and management of ostomy wastes have rejected the use of any device or system that does not collect waste material in an impervious container.

There remains a need for an approach to the management of ostomy-associated wastes that addresses the disadvantages and problems with the conventional devices and approaches known in the prior art.

### SUMMARY OF THE INVENTION

It is among the objects of the present invention to provide an ostomy pad that is simple and easy for an ostomy patient to apply and use.

It is also among the objects of the present invention to provide an ostomy pad that is thin in dimension and eliminates the bulk of conventional ostomy bags or pouches.

It is further among the objects of the present invention to provide an ostomy pad that may be easily and safely disposed of after use, eliminating the need for cleaning associated with conventional ostomy bags or pouches.

It is additionally among the objects of the invention to provide an ostomy pad that is comfortable for a user to wear without skin irritation.

It is a still further object of the invention to provide an ostomy pad that does not significantly limit the physical activity which may be engaged in by a user, allowing a user to enjoy a more normal lifestyle than is possible with conventional ostomy bags or pouches.

Other objects and advantages of the ostomy pad system of the present invention will become apparent from the detailed disclosure and description below.

The ostomy pad of the present invention is, in the preferred embodiment, formed as a highly absorbent relatively thin body of an organic fluff material combined with a liquid absorbing polymer, with an impermeable outer sheet bonded to one face of the absorbent body and a liquid-permeable inner sheet bonded to the opposite face of the absorbent body. The inner and outer sheets are preferably bonded together at their edges to enclose the body material between them. The liquid-permeable inner sheet is preferably formed of a material that will permit the flow of liquid through the sheet to the absorbent body without being itself wet by the liquid, so as to provide a dry layer of material to be received against the skin of a person using the pad. In an alternative design, the inner sheet may be penetrated by an aperture to be received around the stoma, to facilitate entry of waste material into the space between the inner and outer sheets and into contact with the absorbent body.

An adhesive material may be applied to the surface of the inner sheet to removably attach the pad to the skin of a user. As an alternative to, or in addition to the adhesive, the pad may be held in place by an elastic belt or band encircling the user's body and extending over the pad.

The structure and features of the invention and achievement of the objects of the invention will be described in more detail below, with reference to the accompanying drawing figures.

### DESCRIPTION OF THE DRAWING FIGURES

FIG. 1 is a front perspective view of the system of the invention, with an absorbent pad and an encircling elastic band.

FIG. 2 is an elevation view of one configuration of the preferred embodiment of the pad of the invention, showing the outer face.

FIG. 3 is an elevation view of another configuration of the preferred embodiment of the pad of the invention, showing the inner face.

FIG. 4 is an edge view of the preferred embodiment of the pad of the invention.

FIG. 5 is a partial sectioned edge view of the preferred embodiment of the pad of the invention, showing the structure of the body of the pad.

FIG. 6 is a partial front view of the elastic band pad retainer component of the system of the invention.

FIG. 7 is an elevation view of the inner face of an alternative embodiment of the pad of the invention.

FIG. 8 is an elevation view of the inner face of another alternative embodiment of the pad of the invention.

### DESCRIPTION OF THE INVENTION

The present invention provides an approach to the management of ostomy waste material that is a significant departure from the prior art, and that addresses the disadvantages and drawbacks of conventional ostomy appliances. The approach of the present invention highly absorbent materials and selectively permeable materials, formed in a pad structure, to capture and manage both liquid and solid waste materials.

The ostomy pad of the invention, generally identified by reference numeral 10, comprises a highly absorbent relatively thin body 11, preferably formed of an organic fluff material combined with a liquid absorbing polymer, an impermeable outer sheet 12 bonded to one face of the absorbent body, and a liquid-permeable inner sheet 13 bonded to the opposite face of the absorbent body. The inner and outer sheets are preferably bonded together at their outer edges 14 to enclose the body material between them. The liquid-permeable inner sheet is preferably formed of a material that will permit the flow of liquid through the sheet to the absorbent body without being itself wet by the liquid, so as to provide a dry layer of material to be received against the skin of a person using the pad. The specific configuration of the pad, in terms of its overall shape, is not critical to the invention, and can be varied to suit the needs of users and/or to facilitate production. Suitable configurations include oval and rectangular. The pad of the invention may also be trimmed by a user if desired, to provide a customized shape and size. Because of the very high absorbency of the material of the body of the pad, bonding of the outer and inner sheets at the perimeter of the pad, though preferred, is not absolutely necessary. It is, nevertheless, preferred that any trimming of a pad with bonded edges by the user be done along the portion of the edge of the pad that will be positioned above the stoma.

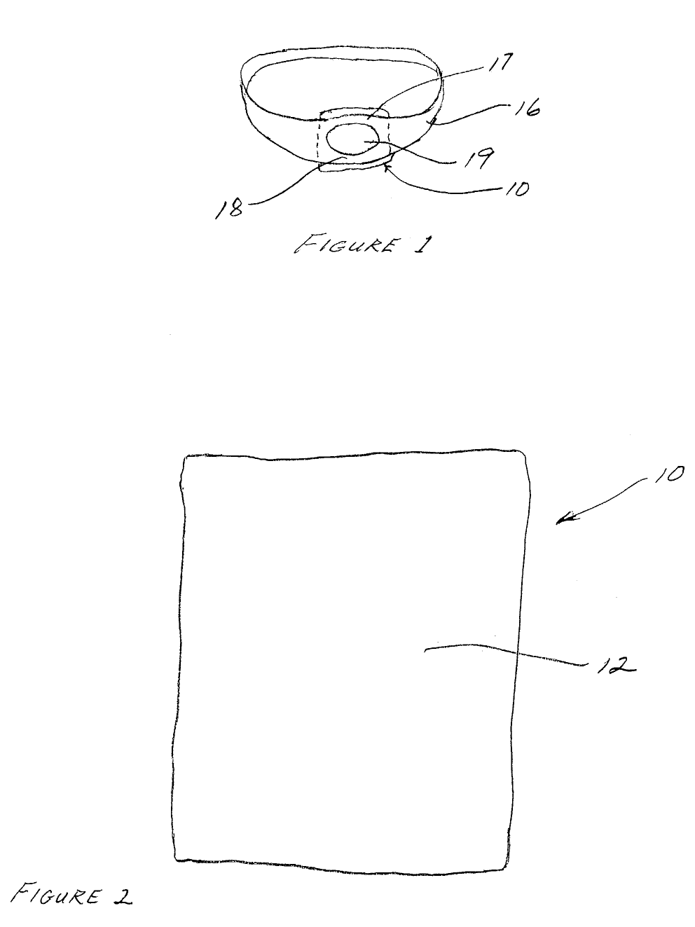

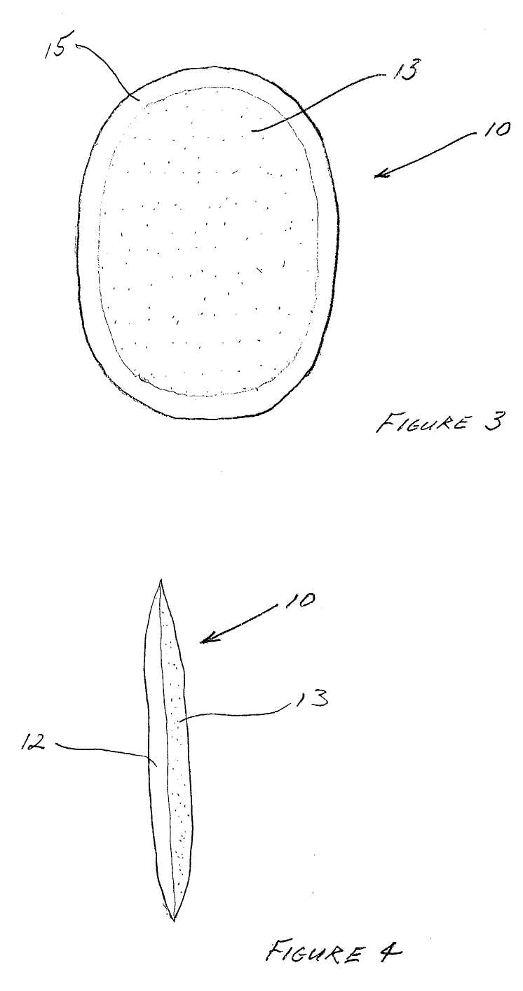

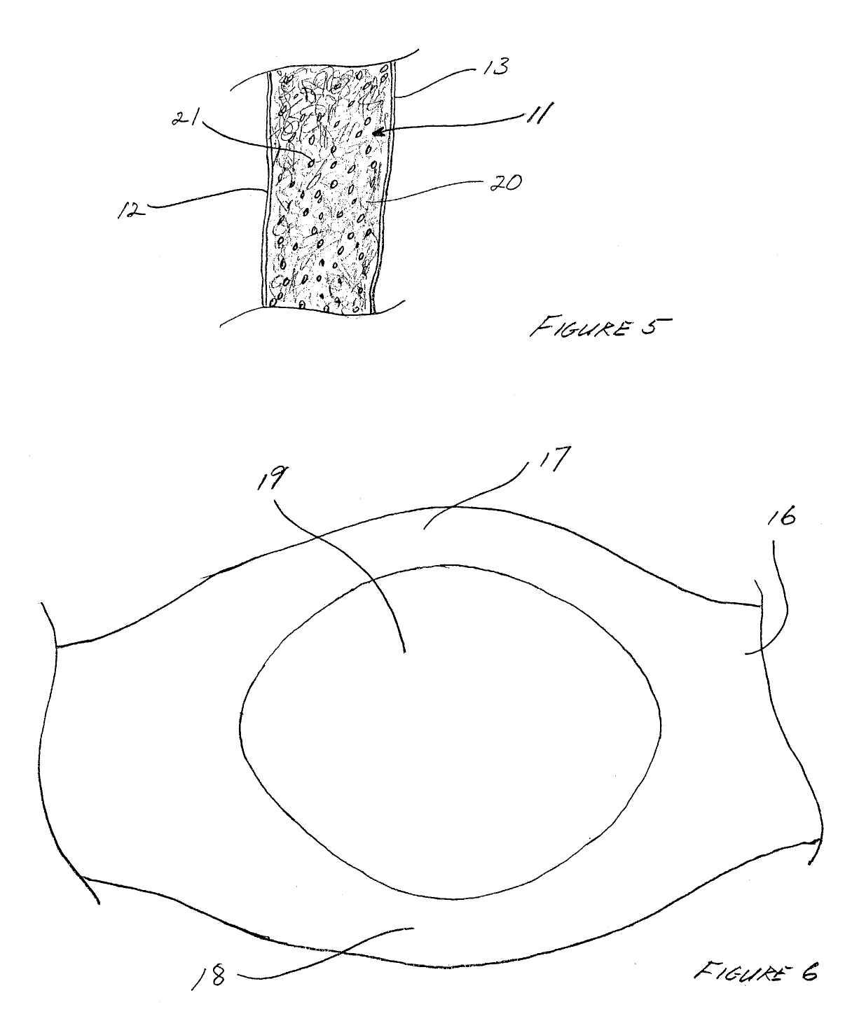

In one embodiment of the pad of the invention, an adhesive material 15, preferably of a hypoallergenic formulation, is applied in an annular band around the periphery of the inner sheet of the pad, as shown in FIG. 3, to adhere the pad to the skin of a user around the stoma. In another embodiment, the pad is free of adhesive and is held in place by an elastic band 16 encircling the torso of a user. The portion of the band, shown in FIGS. 1 and 4, that overlies the pad is divided into two separated portions 17 and 18 with a gap, or stoma aperture 19, between them, so that one of the separated portions will pass above the stoma and the other portion will pass below the stoma. The pad of the invention is placed with one end of the pad under the upper portion 17 of the band and the opposite end of the pad under the lower portion 18 of the band, to secure the pad in place. The sides of the pad are held in place against the skin of the user by the portions of the undivided band on either side of the stoma aperture in the band.

It is preferred that body II of the pad of the invention be formed of a cellulose fluff 20, derived from wood pulp, with a plurality of small grains 21 of a highly absorbent hygroscopic polymer, such as polyacrylate, dispersed through the fluff. The absorbent polymer may also be provided in the form of elongate fibers, if desired. The fluff material is itself highly absorbent, and disperses fluids through the structure of the body of the pad. The polymer material is extremely absorbent, capable of absorbing many times its weight of water. The liquid absorbed by the polymer is bound within the physical structure of the polymer, and does not release under pressure, so liquid absorbed by the polymer cannot be squeezed out of the pad. The structure of the pad and the nature of the absorbent materials comprising the body of the pad eliminates the necessity of assuring the initial formation and maintenance of a complete seal around the stoma, and eliminates the problems with leakage associated with conventional ostomy appliances. The rapid absorption of fluid and the high volume capacity of the pad effectively assure that all fluids that come into contact with the inner surface of the pad are absorbed into the body of the pad.

Although the scope of the invention is not limited to a particular composition, an illustrative composition of the body of the pad is one in which the fluff pulp and binder fibers used to consolidate the fluff comprise approximately 78% of the body by weight, and the highly absorbent polymer comprises approximately 22% by weight. In the pad of the example the outer sheet is formed of a polypropylene material and the inner sheet is formed of polypropylene or polypropylene and polyethylene. The mass per unit area of the pad material, generally referred to as the “weight” of the material, in the example is approximately 300 grams per square meter (gsm). The pad material of the example will absorb approximately 34 grams of water per gram of pad material within a time period of one minute. It is to be expressly understood that the foregoing example is provided for illustration only, and is not in any respect limiting of the scope of the invention.

An odor control agent may also be incorporated into the structure of the body of the pad, in addition to the absorbent materials. Baking soda is a preferred odor control agent, because a small volume or mass is effective in absorbing odors, the granular form of baking soda is easily dispersed through the body of the pad, and baking soda is generally non-irritating to the skin and is generally hypo-allergenic. It is to be understood, however, that other odor control agents may be used if desired.

Because the permeable inner sheet of the pad structure allows liquids to pass through the sheet but does not itself absorb liquid, it presents a dry surface to the skin of a user in contact with the inner sheet. When the pad is used by a urostomy patient, the liquid waste material passes fully through the inner sheet and is completely absorbed by the body of the pad, and only the dry surface of the inner sheet is in direct contact with the skin of the user.

Solid wastes are not directly absorbed by the pad, but the liquid component of the fecal matter is drawn to pass through the permeable inner sheet of the pad and into the body of the pad, where it is absorbed by the polymer and the fluff material, partially drying the fecal matter that is not absorbed into the pad. This drying effect reduces the possibility of skin irritation, makes the pad more comfortable to wear, and facilitates replacement and disposal.

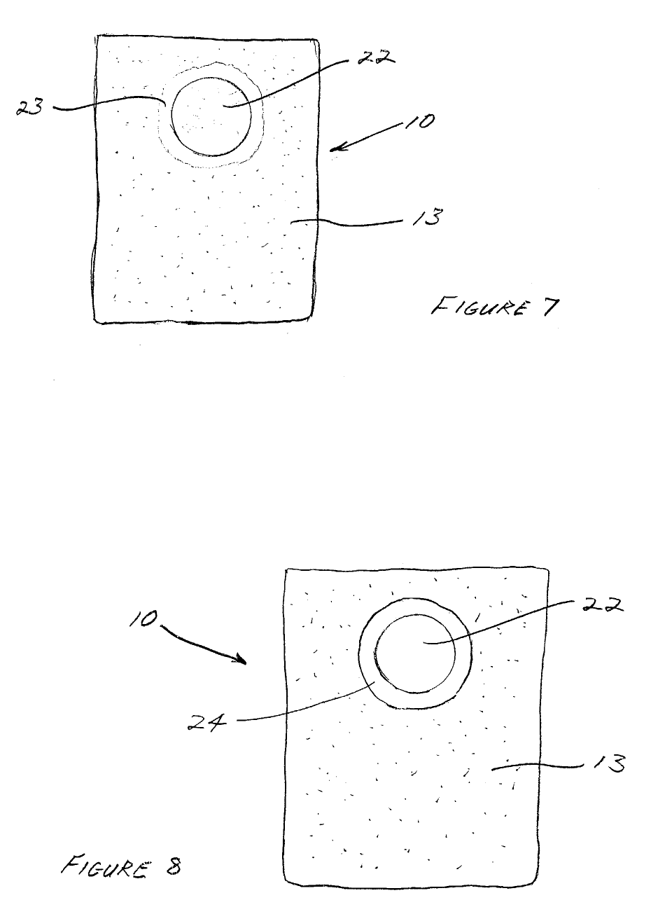

A number of variations may be made in the structure of the primary embodiment of the pad of the invention to provide alternative embodiments. In one such variation, illustrated in FIG. 7, a generally circular waste aperture 22 is formed in permeable inner sheet 13 of the pad, to be aligned with the stoma when the pad is positioned for use. An annular band of adhesive material 23 may be provided around the waste aperture 22, if desired, to adhere the inner sheet of the pad to the skin immediately around the stoma. The pad may be secured to a user with either of the methods described above; an adhesive material at the perimeter of the pad, or an encircling elastic band. With this variation the waste material emitted from the stoma moves directly into the interior of the pad between the inner and outer sheets. As in the primary embodiment, liquids are promptly dispersed through and absorbed by the body of the pad. Solid matter is received in the interior of the pad and isolated from direct contact with the skin by the inner sheet 13 of the pad structure. Any leakage, or waste material that does not pass through the waste aperture 22 to the interior of the pad can permeate the inner sheet and be absorbed by the body of the pad, as described in reference to the primary embodiment.

In a variation of this alternative embodiment, a more rigid annular flange 24 is attached to inner sheet 13 around waste aperture 22, as shown in FIG. 8, to be adhered to the skin around the stoma. Adhesive band 23 is disposed on the face of flange 24 which is received against the skin. Flange 24 may be bonded to inner sheet 13, or it may be provided as a separate component with adhesive on both faces, and attached by the user.

As another variation, or alternative embodiment, inner sheet 13 of the embodiment with waste aperture 22, rather than being permeable as in the preferred embodiment, is formed of an impermeable material, as is outer sheet 12, to fully surround the absorbent body of the pad, except for the waste aperture, with a cover impermeable to liquid. Though not a preferred design, because of the presence of an impermeable sheet next to the skin, which can result in capture of perspiration and exclusion of air, with resulting irritation, and because the absence of a permeable sheet between the skin and the absorbent material makes the creation and maintenance of a secure seal around the stoma more important, this alternative design is within the scope of the invention. Despite its drawbacks, this alternative does have some advantages over the use of conventional ostomy appliances, however. The disadvantage of placing a non-permeable, non-breathing material next to the skin could be offset by covering the outer face of the inner sheet of the pad with a thin layer of material permeable to liquid and air, and preferably capable of absorbing perspiration and any leakage of waste material.

Although the material of the body of the pad is capable of absorbing a large volume of fluid, the dry volume of that material and of the full pad structure, is relatively low and the material is very light. Accordingly, spare pads may be carried by a user very easily and unobtrusively. The pads of the invention may also be disposed of very easily after use, and the process of pad replacement can be easily, quickly, and unobtrusively performed by the user in the privacy of, e.g., a toilet stall in a public restroom.

The foregoing description of preferred and alternative embodiments and variations of the invention is intended to be illustrative and not limiting. It will be understood that the invention is susceptible to various other embodiments and variations, all within the scope of the invention, which is intended to be broadly construed.

1. An absorbent ostomy pad system for use by an ostomy patient for collecting waste emitted from a stoma in the patient's body, comprising
a thin, generally planar, highly flexible absorbent body, with a first face, a second face, and an outer edge, said body to be positioned over the stoma to absorb waste emitted therefrom;
a first, liquid permeable sheet having a first face, a second face, and a continuous outer edge, said first sheet being larger in dimension than said absorbent body, said first sheet disposed on said absorbent body with said first face of said first sheet received against said second face of said absorbent body and with said edge of said first sheet extending outwardly beyond said edge of said absorbent body;
a second, impermeable sheet having a first face, a second face, and a continuous outer edge, said second sheet being larger in dimension than said absorbent body, said second sheet disposed on said absorbent body with said second face of said second sheet received against said first face of said absorbent body and with said edge of said second sheet extending outwardly beyond said edge of said absorbent body, said edge of said second sheet being permanently interconnected to said edge of said first sheet to enclose said absorbent body between said first sheet and said second sheet; and
attachment means for removeably attaching the ostomy pad to the body of an ostomy patient over the stoma.

2. The absorbent ostomy pad system of claim 1, wherein said absorbent body is formed of a cellulose fluff and an absorbent polymer interspersed in said fluff.

3. The absorbent ostomy pad system of claim 1, wherein said attachment means comprises an adhesive material on said second face of said first sheet, for releaseably adhering said pad to the body of an ostomy patient over the stoma.

4. The absorbent ostomy pad system of claim 1, wherein said attachment means comprises an elastic band to encircle the body of an ostomy patient over the stoma with said pad between said band and the patient's body.
